(19) United States
(12) Patent Application Publication
(10) Pub. No.: US 2025/0389944 A1
(43) Pub. Date: Dec. 25, 2025

(54) ILLUMINATION OPTICAL SYSTEM AND ENDOSCOPE

(71) Applicant: FUJIFILM Corporation, Tokyo (JP)

(72) Inventor: Keisuke HARADA, Saitama (JP)

(73) Assignee: FUJIFILM Corporation, Tokyo (JP)

(21) Appl. No.: 19/234,265

(22) Filed: Jun. 10, 2025

(30) Foreign Application Priority Data
Jun. 21, 2024 (JP) 2024-100756

(51) Int. Cl. G02B 23/24; A61B 1/07; G02B 5/02

(52) U.S. Cl. CPC G02B 23/2461; A61B 1/07; G02B 5/0221; G02B 23/2423

(57) ABSTRACT

The illumination optical system is disposed at a distal end of a light guide of an endoscope. A first surface that is a surface of the illumination optical system closest to a light guide side includes a plurality of ring-shaped groove portions that are arranged in a concentric circular shape about an optical axis of the illumination optical system. The groove portions have an inclined surface that narrows a groove width from the light guide side toward an irradiation target side. In the first surface, all surfaces that are perpendicular to the optical axis and adjacent to the inclined surfaces of the groove portions are light diffusion surfaces. The light diffusion surfaces include first light diffusion surfaces that are present between the groove portions adjacent to each other. The illumination optical system satisfies a predetermined conditional expression.

### CROSS-REFERENCE TO RELATED APPLICATIONS

This application claims priority from Japanese Patent Application No. 2024-100756, filed on Jun. 21, 2024, the entire disclosure of which is incorporated herein by reference.

### BACKGROUND

### Technical Field

The technology of the present disclosure relates to an illumination optical system and an endoscope.

### Related Art

In the related art, as an illumination optical system that is disposed at a distal end portion of an insertion portion of an endoscope and illuminates a subject, for example, illumination optical systems described in JP1995-043620A (JP-H07-043620A) and JP2002-244050A are known.

### SUMMARY

In recent years, there has been a demand for an illumination optical system that is configured to be small, has a wide light distribution angle, and has a good transmission efficiency.

The present disclosure provides an illumination optical system that is configured to be small, has a wide light distribution angle, and has a good transmission efficiency, and an endoscope comprising the illumination optical system.

A first aspect of the present disclosure is an illumination optical system that is disposed at a distal end of a light guide of an endoscope, the illumination optical system comprising: a first surface that is a surface of the illumination optical system closest to a light guide side, the first surface including a plurality of ring-shaped groove portions that are arranged in a concentric circular shape about an optical axis of the illumination optical system, in which the groove portions have an inclined surface that narrows a groove width from the light guide side toward an irradiation target side, in the first surface, all surfaces that are perpendicular to the optical axis and adjacent to the inclined surfaces of the groove portions are light diffusion surfaces, the light diffusion surfaces include first light diffusion surfaces that are present between the groove portions adjacent to each other, and in a case where a radial length of the first light diffusion surface is denoted by C and a distance from the optical axis to a position of an outermost diameter of the groove portion on a most outer diameter side is denoted by A,

Conditional Expression (1) is satisfied, which is represented by

$$0.01 < C / A < 0.3 . \qquad ( 1 )$$

A second aspect of the present disclosure is the illumination optical system according to the first aspect, in which the light diffusion surfaces include a second light diffusion surface adjacent to the inclined surface on an outer diameter side of the groove portion on the most outer diameter side.

A third aspect of the present disclosure is the illumination optical system according to the second aspect, in which, in a case where a radius of the illumination optical system is denoted by B, a distance in a direction of the optical axis from the second light diffusion surface to an intersection between the optical axis and a surface of the illumination optical system closest to the irradiation target side is denoted by L, and an angle formed between the inclined surface on the outer diameter side of the groove portion on the most outer diameter side and the surface perpendicular to the optical axis is θ,

Conditional Expression (2) is satisfied, which is represented by

$$0.5 < ( B - A ) / ( L \times tan θ ) < 1.5 . \qquad ( 2 )$$

A fourth aspect of the present disclosure is the illumination optical system according to the second aspect, in which, in a case where a distance in a direction of the optical axis from the second light diffusion surface to an intersection between the optical axis and a surface of the illumination optical system closest to the irradiation target side is denoted by L, and a distance in the direction of the optical axis from a deepest portion of the groove portion closest to the optical axis to the intersection is denoted by K,

Conditional Expression (3) is satisfied, which is represented by

$$0.5 < K / L < 0 \text{.88} . \qquad ( 3 )$$

A fifth aspect of the present disclosure is the illumination optical system according to the second aspect, in which, in a case where a distance in a direction of the optical axis from the second light diffusion surface to an intersection between the optical axis and a surface of the illumination optical system closest to the irradiation target side is denoted by L, a distance in the direction of the optical axis from a deepest portion of the groove portion closest to the optical axis to the intersection is denoted by K, and an angle formed between the inclined surface of the groove portion closest to the optical axis and the surface perpendicular to the optical axis is denoted by φ,

Conditional Expression (4) is satisfied, which is represented by

$$0.104 < ( L - K ) / ( A \times tan φ ) < 0.4 . \qquad ( 4 )$$

A sixth aspect of the present disclosure is the illumination optical system according to the first aspect, in which, in a case where a radius of an emission end surface of the light guide is denoted by G,

Conditional Expression (5) is satisfied, which is represented by

$$1 < G / A < 1.8 . \qquad ( 5 )$$

A seventh aspect of the present disclosure is the illumination optical system according to the first aspect, in which, in a case where an angle formed between the inclined surface on an outer diameter side of the groove portion on the most outer diameter side and the surface perpendicular to the optical axis is denoted by θ, an angle formed between the inclined surface on an optical axis side of the groove portion on the most outer diameter side and the surface perpendicular to the optical axis is denoted by ω, and units of θ and ω are degrees,

Conditional Expression (6) is satisfied, which is represented by

$$20 < \vert ω - θ \vert < 50. \qquad ( 6 )$$

An eighth aspect of the present disclosure is the illumination optical system according to the first aspect, in which, in a case where a radius of the illumination optical system is denoted by B,

Conditional Expression (7) is satisfied, which is represented by

$$1.2 < B / A < 2.5 . \qquad ( 7 )$$

A ninth aspect of the present disclosure is an endoscope comprising the illumination optical system according to any one of the first to eighth aspects.

In the present specification, “consist of” or “consisting of” is intended to mean that a lens that substantially does not have optical power, an optical element other than a lens, such as a stop, a filter, and a cover glass, a lens flange, a lens barrel, and the like may be included in addition to the illustrated constituents.

According to the present disclosure, it is possible to provide an illumination optical system that is configured to be small, has a wide light distribution angle, and has a good transmission efficiency, and an endoscope including the illumination optical system.

### BRIEF DESCRIPTION OF THE DRAWINGS

FIG. 1 is a cross-sectional view showing a configuration of an illumination optical system according to an embodiment corresponding to an illumination optical system of Example 1.

FIG. 2 is a diagram showing a configuration in a surface perpendicular to an optical axis of the illumination optical system of FIG. 1.

FIG. 3 is a cross-sectional view showing a configuration and optical paths of the illumination optical system of FIG. 1.

FIG. 4 is a diagram for describing a Comparative Example.

FIG. 5 is a diagram for describing symbols of Conditional Expressions in the illumination optical system of FIG. 1.

FIG. 6 is a graph showing a light distribution characteristic of the illumination optical system of Example 1.

FIG. 7 is a cross-sectional view showing a configuration and optical paths of an illumination optical system of Example 2.

FIG. 8 is a diagram showing a configuration in a surface perpendicular to an optical axis of the illumination optical system of Example 2.

FIG. 9 is a graph showing a light distribution characteristic of the illumination optical system of Example 2.

FIG. 10 is a cross-sectional view showing a configuration and optical paths of an illumination optical system of Example 3.

FIG. 11 is a diagram showing a configuration in a surface perpendicular to an optical axis of the illumination optical system of Example 3.

FIG. 12 is a graph showing a light distribution characteristic of the illumination optical system of Example 3.

FIG. 13 is a cross-sectional view showing a configuration of an illumination optical system of Example 4.

FIG. 14 is a cross-sectional view showing a configuration and optical paths of the illumination optical system of Example 4.

FIG. 15 is a diagram showing a configuration in a surface perpendicular to an optical axis of the illumination optical system of Example 4.

FIG. 16 is a diagram for describing symbols of Conditional Expressions in the illumination optical system of Example 4.

FIG. 17 is a graph showing a light distribution characteristic of the illumination optical system of Example 4.

FIG. 18 is a cross-sectional view showing a configuration and optical paths of an illumination optical system of Example 5.

FIG. 19 is a diagram showing a configuration in a surface perpendicular to an optical axis of the illumination optical system of Example 5.

FIG. 20 is a graph showing a light distribution characteristic of the illumination optical system of Example 5.

FIG. 21 is a cross-sectional view showing a configuration and optical paths of the illumination optical system of Example 6.

FIG. 22 is a diagram showing a configuration in a surface perpendicular to an optical axis of an illumination optical system of Example 6.

FIG. 23 is a graph showing a light distribution characteristic of the illumination optical system of Example 6.

FIG. 24 is a schematic configuration view of an endoscope according to the embodiment.

### DETAILED DESCRIPTION

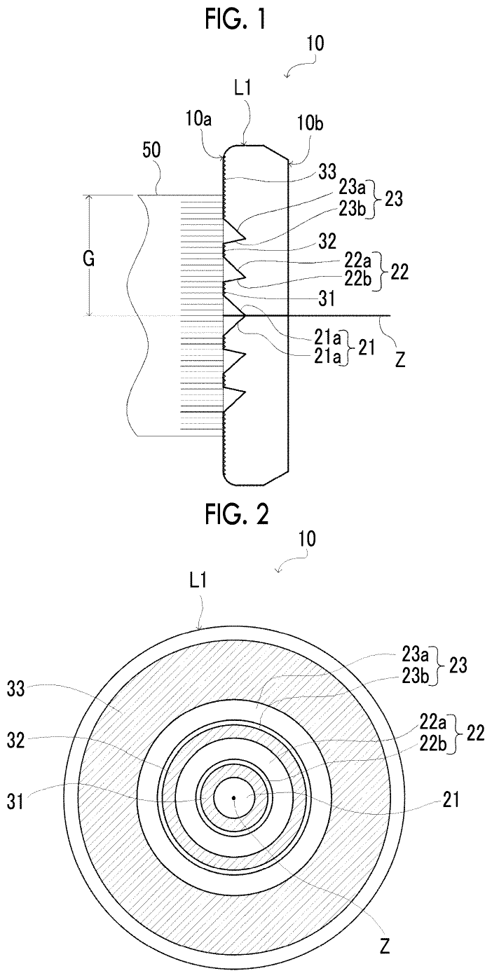

Embodiments of the present disclosure will be described in detail below with reference to the drawings. FIG. 1 shows a configuration in a cross section including an optical axis Z of an illumination optical system 10 according to an embodiment of the present disclosure. The illumination optical system 10 shown in FIG. 1 corresponds to Example 1 to be described later.

The illumination optical system 10 is an optical system that is disposed at a distal end of a light guide 50 of an endoscope. The light guide 50 consists of a bundle fiber in which a plurality of optical fibers are bundled, and emits light, which is emitted from a light source (not shown), to the illumination optical system 10. That is, the light emitted from the light source is incident on the illumination optical system 10 via the light guide 50, is emitted from the illumination optical system 10, and serves as illumination light. In a case where the illumination optical system 10 is disposed at a distal end portion of an insertion portion of the endoscope, an irradiation target (not shown), which is an object to be observed, is illuminated with the illumination light. FIG. 1 shows a cross-sectional view of the illumination optical system 10 including an optical axis Z, and a left side is a light source side and a right side is an irradiation target side.

Among the surfaces of the illumination optical system 10, a first surface 10a is a surface closest to the light guide 50 side of the illumination optical system 10. The first surface 10a includes a plurality of groove portions having a ring shape arranged in a concentric circular shape about the optical axis Z. The groove portion has an inclined surface that narrows a groove width from the light guide side toward the irradiation target side. FIG. 2 shows a configuration in a surface perpendicular to an optical axis Z of the illumination optical system 10 of FIG. 1, viewed from the light source side.

For example, the illumination optical system 10 shown in FIG. 1 consists of one optical element L1. However, the illumination optical system of the present disclosure may include a plurality of elements, and may include an optical element having no refractive power, for example, a plane-parallel plate. The illumination optical system 10 in FIG. 1 has a rotationally symmetric configuration with the optical axis Z as a rotation axis. In FIG. 1, in order to avoid complication of the drawing, the reference numerals are omitted for a part of the portion below the optical axis Z.

For example, the first surface 10a of the illumination optical system 10 in FIG. 1 includes three groove portions 21, 22, and 23 in order from the optical axis side to the outer diameter side. The groove portion 21 on the optical axis has a V-shaped cross section and has an inclined surface 21a. The groove portion 22 has a substantially V-shaped cross section, and has an inclined surface 22a on the outer diameter side and an inclined surface 22b on the optical axis side. The groove portion 23 has a substantially V-shaped cross section, and has an inclined surface 23a on the outer diameter side and an inclined surface 23b on the optical axis side. By setting the shape of each groove portion in the cross section including the optical axis Z to a V-shape or a substantially V-shape, there is an advantage in widening the light distribution angle.

In the following description, for convenience of description, the groove portions 21, 22, and 23 will be simply referred to as “groove portions” unless otherwise necessary to distinguish therebetween. In addition, in a case where it is not necessary to distinguish and describe the inclined surfaces of each of the groove portions, the inclined surfaces are simply referred to as “inclined surfaces”.

A surface perpendicular to the optical axis Z and adjacent to the inclined surface of the groove portion in the first surface 10a is configured to be a light diffusion surface. The ray that has passed through the light diffusion surface is projected onto an irradiation target in a state where the light spreads widely due to the diffusion. Accordingly, by including the light diffusion surface in the first surface 10a, there is an advantage in widening the light distribution angle.

In the description of the present specification, “perpendicular” indicates being substantially perpendicular including an error generally allowed in the technical field to which the technology of the present disclosure belongs, in addition to being completely perpendicular. In addition, the above-described “surface perpendicular to the optical axis Z” refers to a shape in comparison with the groove portion, which is not microscopic but macroscopic.

The light diffusion surface is a surface having a function of diffusing light. The light diffusion surface may be a roughened surface having fine unevenness, and may be, for example, a surface subjected to a sandblasting treatment by polishing. In addition, the light diffusion surface may be a surface on which a layer containing a substance having a light diffusion function, such as glass beads, is provided. The surface roughness of the light diffusion surface having fine unevenness can be typically set to a few μm or less in terms of arithmetic average roughness Ra, and is preferably 0.3 to 0.7 μm.

In the example of FIG. 1, a light diffusion surface 31 is formed adjacent to the inclined surface 21a and the inclined surface 22b between the groove portion 21 and the groove portion 22 which are adjacent to each other, and a light diffusion surface 32 is formed adjacent to the inclined surface 22a and the inclined surface 23b between the groove portion 22 and the groove portion 23 which are adjacent to each other. In addition, the light diffusion surface 33 is formed on the outer diameter side adjacent to the inclined surface 23a of the groove portion 23 on the most outer diameter side. The light diffusion surfaces 31 and 32 are examples of first light diffusion surfaces of the technology of the present disclosure. The light diffusion surface 33 is an example of a second light diffusion surface of the technology of the present disclosure. In the following description, for convenience of description, the light diffusion surfaces 31, 32, and 33 will be simply referred to as “light diffusion surface” unless otherwise necessary to distinguish therebetween.

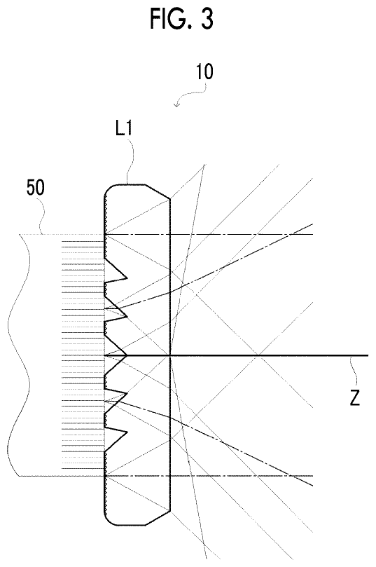

In FIG. 3, the optical path of the illumination optical system 10 of FIG. 1 having the above-described configuration is shown by a solid line and a one-dot chain line. FIG. 3 shows a state of rays in a case where a plurality of rays emitted from a plurality of points on an emission end surface of the light guide 50 are incident on the illumination optical system 10. As shown in FIG. 3, the illumination optical system 10 in FIG. 1 realizes a wide light distribution angle.

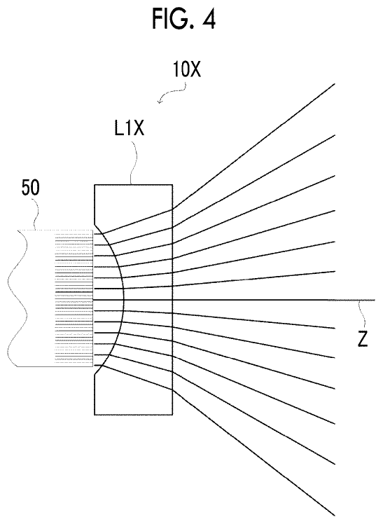

Hereinafter, the action and effect of the illumination optical system 10 of the present example will be described in detail. By providing the groove portion having the inclined surface, the rays emitted from the light guide 50 can be emitted from the illumination optical system 10 as rays having a large spread angle. In the illumination optical system 10X consisting of a negative lens of a Comparative Example of FIG. 4 described below, the spread angle of the ray emitted from the vicinity of the center portion of the light guide 50 is small. On the other hand, in the illumination optical system 10 of the present example, even a ray emitted from the vicinity of the center portion of the light guide 50 can be emitted from the illumination optical system 10 as a ray having a large spread angle.

In the present example, by providing a plurality of ring-shaped groove portions, the thickness of the illumination optical system 10 in the optical axis direction can be reduced as compared with the configuration described in JP2002-244050A. In addition, since a plurality of ring-shaped groove portions are provided, the range of the groove portion on the first surface 10a can be reduced. Therefore, there is an advantage in achieving reduction in diameter of the illumination optical system 10. In the present example, even in a case where the range of the groove portion is reduced, the light diffusion surface is provided, and thus the light distribution angle can be kept wide. In particular, in the present example, since the light diffusion surface 33 is provided adjacent to the inclined surface 23a of the groove portion 23 on the most outer diameter side, even in a case where the range of the groove portion is reduced, the light distribution angle can be kept wide. As described above, according to the illumination optical system 10 of the present example, it is possible to achieve both size reduction and a wide light distribution angle.

In addition, the present example is advantageous in that, since the light diffusion surface is provided between the groove portions, the unevenness of the light distribution is reduced while the light distribution angle is widened as described below. Since the clad not emitting light, gaps between the fibers, and the core emitting light are arranged on the same surface on the emission end surface of the light guide 50 consisting of a bundle fiber, the emission end surface includes dark portions and bright portions. In a case where an image of the emission end surface of the light guide 50 is formed on the irradiation target, a pattern of light and shade is projected onto the irradiation target, and a significant unevenness of the light distribution occurs. In a case where the projection image of the pattern of light and shade is clear, there is a concern that the observation of the irradiation target may be hindered. In this regard, in the present example in which the light diffusion surface is provided, the light is diffused by the light diffusion surface, and thus the occurrence of the projection image of the pattern of light and shade on the irradiation target can be suppressed, and thus the unevenness of the light distribution can be suppressed.

FIG. 4 shows a state of rays in an illumination optical system 10X in a case where the illumination optical system 10 of FIG. 1 is temporarily replaced with a lens L1X having neither a groove portion nor a light diffusion surface as a Comparative Example. The lens LIX in FIG. 4 is a negative lens of which the surface on the light guide side is a concave surface. In the example of FIG. 4, the spread angle of the ray emitted from the vicinity of the center portion of the light guide 50 is small, and the spread angle of the ray tends to increase as the emission position is farther from the center portion toward the outer diameter side. In this configuration, in a case where the outer diameter of the lens LIX is reduced for size reduction, there is a concern that rays having a large emission angle may be blocked. That is, in the example of FIG. 4, it is difficult to achieve both size reduction and a wide light distribution angle.

In addition, in the lens LIX that does not have the light diffusion surface, the degree of diffusion of the ray is weak, and thus the ray is projected onto the irradiation target in a state where the spread of the light is small. Accordingly, in the illumination optical system 10X, it is difficult to keep the light distribution angle wide as compared with the illumination optical system 10 of the present example, and a projection image of a pattern of light and shade caused by the dark portion and the bright portion of the light guide 50 described above is generated on the irradiation target, and the possibility that unevenness of the light distribution occurs increases.

Further, since the light from the light guide actually has a spread angle, in a case where the light emitted from a position close to an outer periphery of the emission end surface of the light guide passes through the concave surface of the lens LIX, light leak may occur. In order to reduce the size, the diameter of the concave surface of the lens LIX may be configured to be the same as the diameter of the emission end surface of the light guide 50, and in such a configuration, the light leak is likely to occur. The occurrence of light leak reduces the light transmission efficiency. On the other hand, in the illumination optical system 10 of the present example in FIG. 1, since the light diffusion surface 33 is provided adjacent to the inclined surface 23a of the groove portion 23 on the most outer diameter side, the decrease in the light transmission efficiency due to the above-described light leak can be suppressed.

Next, a preferred configuration and a possible configuration of the conditional expression will be described. In the following description related to the conditional expressions, in order to avoid redundant description, the same symbol will be used for the same definition to partially omit duplicate descriptions of the symbol. In addition, in the following description of the conditional expressions, the light diffusion surface between adjacent groove portions of the light diffusion surfaces is referred to as a first light diffusion surface, and the light diffusion surface adjacent to the inclined surface on the outer diameter side of the groove portion on the most outer diameter side is referred to as a second light diffusion surface.

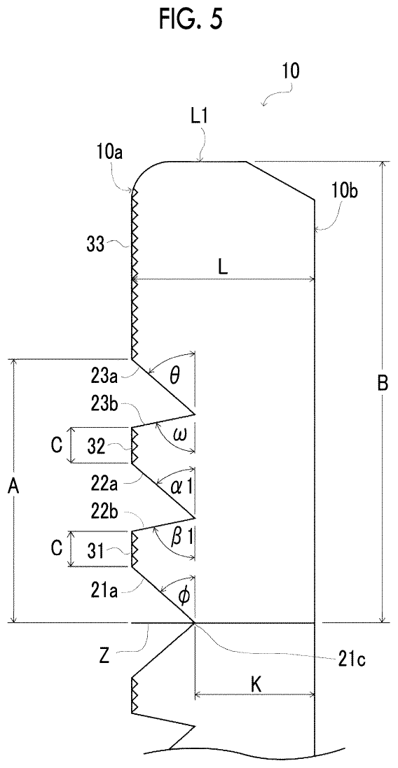

It is preferable that the illumination optical system 10 satisfies the following conditional expression (1). Here, the length of the first light diffusion surface in the radial direction is denoted by C. A distance from the optical axis Z to a position of the outermost diameter of the groove portion on the most outer diameter side is denoted by A. The radial direction is a direction perpendicular to the optical axis Z. In a case where there are a plurality of first light diffusion surfaces, an average value of the radial lengths of the plurality of first light diffusion surfaces is denoted by C. FIG. 5 shows a partially enlarged view of the illumination optical system 10 of FIG. 1, and shows the length C and the distance A as an example. In FIG. 5, the illustration of some reference numerals is omitted. By not allowing the corresponding value of Conditional Expression (1) to be equal to or less than the lower limit value thereof, the spread angle of the emitted light can be increased. Therefore, it is easy to secure a wide light distribution angle. By not allowing the corresponding value of Conditional Expression (1) to be equal to or greater than the upper limit value, it is easy to achieve reduction in size, and there is an advantage in improving the light transmission efficiency.

$$0.01 < C / A < 0.3 \qquad ( 1 )$$

In order to obtain more favorable characteristics, the lower limit value of Conditional Expression (1) is more preferably 0.015, still more preferably 0.02, and even more preferably 0.025. In order to obtain more favorable characteristics, the upper limit value of Conditional Expression (1) is more preferably 0.25, still more preferably 0.2, and even more preferably 0.15.

It is preferable that the illumination optical system 10 satisfies the following conditional expression (2). Here, it is assumed that the radius of the illumination optical system 10 is B. A distance in the direction of the optical axis Z from the second light diffusion surface to an intersection of the optical axis Z and a surface 10b of the illumination optical system 10 closest to the irradiation target side is denoted by L. An angle between the inclined surface on the most outer diameter side of the groove portion on the outer diameter side and a surface perpendicular to the optical axis Z is represented by θ. 0 is an angle on an acute angle side instead of an angle on an obtuse angle side. For example, FIG. 5 shows the radius B, the distance L, and the angle θ. By not allowing the corresponding value of Conditional Expression (2) to be equal to or less than the lower limit value thereof, there is an advantage in improving the light transmission efficiency. By not allowing the corresponding value of Conditional Expression (2) to be equal to or greater than the upper limit value thereof, the spread angle of the emitted light can be increased. Therefore, it is easy to secure a wide light distribution angle.

$$0.5 < ( B - A ) / ( L \times tan θ ) < 1.5 \qquad ( 2 )$$

In order to obtain more favorable characteristics, the lower limit value of Conditional Expression (2) is more preferably 0.53, still more preferably 0.56, and even more preferably 0.6. In order to obtain more favorable characteristics, the upper limit value of Conditional Expression (2) is more preferably 1.35, still more preferably 1.25, and even more preferably 1.1.

It is preferable that the illumination optical system 10 satisfies the following Conditional Expression (3). Here, a distance in the direction of the optical axis Z from the deepest portion of the groove portion closest to the optical axis Z to the intersection between the optical axis Z and the surface 10b of the illumination optical system 10 closest to the irradiation target side is denoted by K. In a case where the groove portion is positioned on the optical axis, the groove portion positioned on the optical axis is defined as the groove portion closest to the optical axis Z. For example, FIG. 5 shows the deepest portion 21c and the distance K. In the example of FIG. 5, the deepest portion 21c is located on the optical axis. By not allowing the corresponding value of Conditional Expression (3) to be equal to or less than the lower limit value thereof, the thickness is not excessively reduced, and thus the breakage is less likely to occur. By not allowing the corresponding value of Conditional Expression (3) to be equal to or greater than the upper limit value thereof, it is possible to suppress the light intensity in the vicinity of the center from being excessively high as compared with other portions. Therefore, it is easy to make the light intensity distribution of the illumination light emitted from the illumination optical system 10 uniform.

$$0.5 < K / L < 0.88 \qquad ( 3 )$$

In order to obtain more favorable characteristics, the lower limit value of Conditional Expression (3) is more preferably 0.53, still more preferably 0.57, and even more preferably 0.6. In order to obtain more favorable characteristics, the upper limit value of Conditional Expression (3) is more preferably 0.85, still more preferably 0.82, and even more preferably 0.8.

In a case where an angle between the inclined surface of the groove portion closest to the optical axis Z and the surface perpendicular to the optical axis Z is denoted by φ, it is preferable that the illumination optical system 10 satisfies Conditional Expression (4). φ is an angle on an acute angle side instead of an angle on an obtuse angle side. For example, FIG. 5 shows the angle φ. By not allowing the corresponding value of Conditional Expression (4) to be equal to or less than the lower limit value thereof, it is possible to suppress the light intensity in the vicinity of the center from being excessively high as compared with other portions. Therefore, it is easy to make the light intensity distribution of the illumination light emitted from the illumination optical system 10 uniform. By not allowing the corresponding value of Conditional Expression (4) to be equal to or greater than the upper limit value thereof, the thickness is not excessively reduced, and thus the breakage is less likely to occur.

$$0.104 < ( L - K ) / ( A \times tan φ ) < 0.4 \qquad ( 4 )$$

In order to obtain more favorable characteristics, the lower limit value of Conditional Expression (4) is more preferably 0.11, still more preferably 0.12, and even more preferably 0.13. In order to obtain more favorable characteristics, the upper limit value of Conditional Expression (4) is more preferably 0.37, still more preferably 0.33, and even more preferably 0.3.

In a case where a radius of the emission end surface of the light guide is denoted by G, it is preferable that the illumination optical system 10 satisfies Conditional Expression (5). For example, FIG. 1 shows the radius G. By not allowing the corresponding value of Conditional Expression (5) to be equal to or less than the lower limit value, it is easy to achieve reduction in size, and there is an advantage in improving the light transmission efficiency. By not allowing the corresponding value of Conditional Expression (5) to be equal to or greater than the upper limit value thereof, the spread angle of the emitted light can be increased. Therefore, it is easy to secure a wide light distribution angle.

$$1 < G / A < 1.8 \qquad ( 5 )$$

In order to obtain more favorable characteristics, the lower limit value of Conditional Expression (5) is more preferably 1.06, still more preferably 1.08, and even more preferably 1.1. In order to obtain more favorable characteristics, the upper limit value of Conditional Expression (5) is more preferably 1.7, still more preferably 1.6, and even more preferably 1.45.

It is preferable that the illumination optical system 10 satisfies the following Conditional Expression (6). Here, it is assumed that an angle formed between the inclined surface on the optical axis side of the groove portion on the most outer diameter side and the surface perpendicular to the optical axis Z is denoted by ω. The units of 0 and ω are degrees. ω is an angle on an acute angle side instead of an angle on an obtuse angle side. For example, FIG. 5 shows the angle ω. By not allowing the corresponding value of Conditional Expression (6) to be equal to or less than the lower limit value thereof, it is possible to suppress the light intensity in the vicinity of the center from being excessively high as compared with other portions. Therefore, it is easy to make the light intensity distribution of the illumination light emitted from the illumination optical system 10 uniform. By not allowing the corresponding value of Conditional Expression (6) to be equal to or greater than the upper limit value thereof, there is an advantage in improving the light transmission efficiency.

$$20 < \vert ω - θ \vert < 50 \qquad ( 6 )$$

In order to obtain more favorable characteristics, the lower limit value of Conditional Expression (6) is more preferably 21, still more preferably 22, and even more preferably 24. In order to obtain more favorable characteristics, the upper limit value of Conditional Expression (6) is more preferably 48, still more preferably 46, and even more preferably 44.

It is preferable that the illumination optical system 10 satisfies the following Conditional Expression (7). By not allowing the corresponding value of Conditional Expression (7) to be equal to or less than the lower limit value, it is easy to achieve reduction in size, and there is an advantage in improving the light transmission efficiency. By not allowing the corresponding value of Conditional Expression (7) to be equal to or greater than the upper limit value thereof, the spread angle of the emitted light can be increased. Therefore, it is easy to secure a wide light distribution angle.

$$1.2 < B / A < 2.5 \qquad ( 7 )$$

In order to obtain more favorable characteristics, the lower limit value of Conditional Expression (7) is more preferably 1.35, still more preferably 1.45, and even more preferably 1.55. In order to obtain more favorable characteristics, the upper limit value of Conditional Expression (7) is more preferably 2.3, still more preferably 2.15, and even more preferably 2.

The preferable configurations and available configurations described above, also including configurations related to conditional expressions, may be optionally combined without contradiction, and it is preferable to selectively adopt configurations in accordance with required specifications as appropriate.

As an example, a preferred aspect of the illumination optical system according to the embodiment of the present disclosure is an illumination optical system that is disposed at a distal end of a light guide of an endoscope, the illumination optical system comprising: a first surface that is a surface of the illumination optical system closest to a light guide side, the first surface including a plurality of ring-shaped groove portions that are arranged in a concentric circular shape about an optical axis Z of the illumination optical system, in which the groove portions have an inclined surface that narrows a groove width from the light guide side toward an irradiation target side, in the first surface, all surfaces that are perpendicular to the optical axis Z and adjacent to the inclined surfaces of the groove portions are light diffusion surfaces, the light diffusion surfaces include first light diffusion surfaces that are present between the groove portions adjacent to each other, and the illumination optical system satisfies Conditional Expression (1).

Next, examples of the illumination optical system according to the embodiment of the present disclosure will be described with reference to the drawings. The reference numerals added to the optical elements in the views of each example are used independently for each example in order to avoid complication of description and drawings due to an increase in number of digits of the reference numerals. Accordingly, a common reference numeral provided in the drawings of different examples does not necessarily indicate a common configuration.

### Example 1

The configuration of the illumination optical system of Example 1 is shown in FIGS. 1 and 2, the optical path of the illumination optical system of Example 1 is shown in FIG. 3, and the method of showing the configuration is as described above. Therefore, the repeated description will be partially omitted. The illumination optical system of Example 1 consists of one optical element L1. The first surface 10a closest to the light guide 50 side includes the groove portions 21, 22, and 23 and the light diffusion surfaces 31, 32, and 33. The groove portions 21, 22, and 23 are ring-shaped grooves disposed in a concentric circular shape about the optical axis Z, and have a V-shape or a substantially V-shape in a cross section including the optical axis Z. The groove portion 21 has an inclined surface 21a. The groove portion 22 has an inclined surface 22a on the outer diameter side and an inclined surface 22b on the optical axis side. The groove portion 23 has an inclined surface 23a on the outer diameter side and an inclined surface 23b on the optical axis side. A light diffusion surface 31 is provided between the groove portion 21 and the groove portion 22, a light diffusion surface 32 is provided between the groove portion 22 and the groove portion 23, and a light diffusion surface 33 is provided on the outer diameter side adjacent to the groove portion 23. The light diffusion surfaces 31, 32, and 33 are surfaces perpendicular to the optical axis Z, and the positions of the light diffusion surfaces 31, 32, and 33 in the optical axis direction are the same. An outline of the illumination optical system of Example 1 is as described above. In the illumination optical system of Example 1, in a cross section including the optical axis Z, the vicinity of the deepest portion of the groove portion 21 has an apex angle, and the vicinity of the deepest portion of the groove portions 22 and 23 has an acute angle shape.

Regarding the illumination optical system of Example 1, construction data is shown in Table 1, and specifications are shown in Table 2. The table of the construction data is described as follows. The column of Sn shows the surface number given in order from the light guide side to the irradiation target side. However, since the first surface 10a includes the light diffusion surface and the groove portion, the light diffusion surface and the deepest portion of the groove portion are described as different surfaces in the table for convenience. The surface of the surface number 1 corresponds to the surfaces of the light diffusion surfaces 31, 32, and 33. The surface of the surface number 2 corresponds to the deepest portion 21c (see FIG. 5) of the groove portion 21 on the optical axis. In the illumination optical system consisting of one optical element L1, the surface with the surface number 3 corresponds to the surface 10b of the illumination optical system closest to the irradiation target side. The column of R shows the curvature radius of each surface other than the surface numbers 1 and 2. The column of D shows the surface spacing in the optical axis direction between each surface and the next surface on the irradiation target side. The column of Nd shows a refractive index with respect to a d line (wavelength of 587.56 nanometers (nm)). The column of vd shows the Abbe number based on the d line.

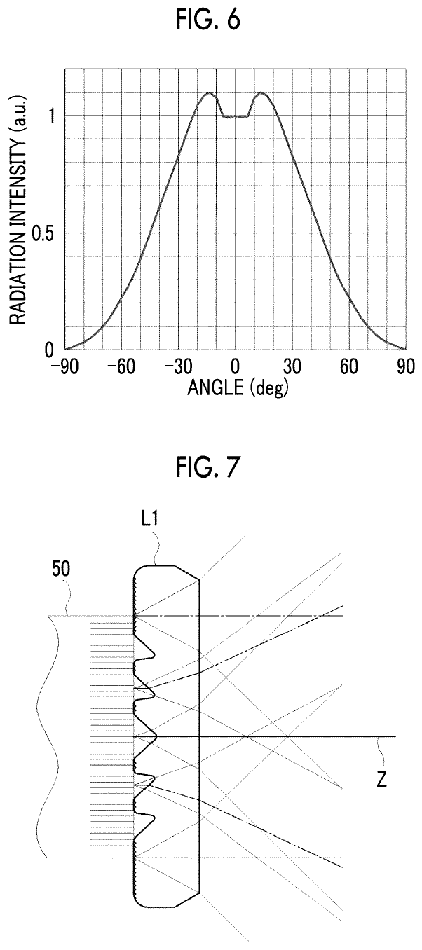

Table 2 shows each value used in the above-described Conditional Expressions and the values of the angles α1 and β1 shown in FIG. 5. α1 is an angle formed between the inclined surface 22a of the groove portion 22 on the outer diameter side and the surface perpendicular to the optical axis Z. β1 is an angle formed between the inclined surface 22b of the groove portion 22 on the optical axis side and the surface perpendicular to the optical axis Z. FIG. 6 shows a graph of the light distribution characteristics of the illumination optical system of Example 1. In FIG. 6, the lateral axis represents an angle from the optical axis Z, and the vertical axis represents a radiation intensity. The half-width at half-maximum of the graph of FIG. 6 is shown in the lowest column of Table 2.

In the data of each table, a millimeter unit is used for lengths, and a degree unit is used for angles. However, since the optical system can also be proportionally enlarged or proportionally reduced to be used, other appropriate units can also be used. Each table below shows numerical values rounded to predetermined digits.

TABLE 1
Example 1

| Sn | R | D | Nd | νd |
|---|---|---|---|---|
| 1 | | 0.0800 | | |
| 2 | | 0.1500 | 1.4585 | 67.82 |
| 3 | ∞ | | | |

TABLE 2
Example 1

| | |
|---|---|
| A | 0.3424 |
| B | 0.600 |
| C | 0.0462 |
| G | 0.425 |
| K | 0.1500 |
| L | 0.230 |
| θ | 48 |
| ω | 78 |
| φ | 48 |
| α1 | 48 |
| β1 | 78 |
| Half-width at half-maximum | 45.0 |

Symbols, meanings, description methods, and methods of showing each data related to Example 1 are basically the same for the following examples unless otherwise specified. Thus, duplicate descriptions will be omitted below.

### Example 2

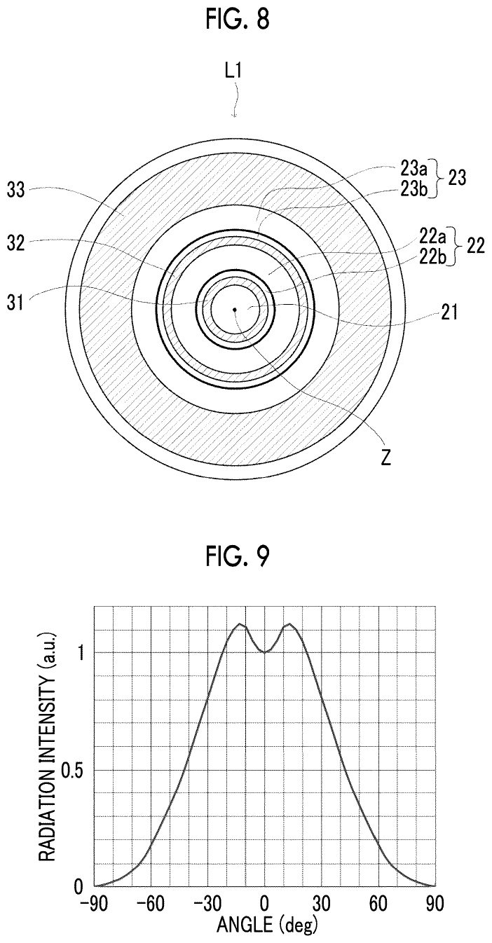

FIG. 7 is a cross-sectional view showing a configuration and optical paths of an illumination optical system of Example 2. FIG. 8 shows a configuration in a surface perpendicular to an optical axis Z of the illumination optical system of Example 2, viewed from the light source side. The illumination optical system of Example 2 has the same configuration as the outline of the illumination optical system of Example 1. In FIG. 7, some reference numerals are omitted in order to avoid complication of the drawing. In the illumination optical system of Example 2, the vicinity of the deepest portion of the groove portions 21, 22, and 23 has a shape having a curvature.

Regarding the illumination optical system of Example 2, Table 3 shows construction data, Table 4 shows specifications, and FIG. 9 shows a graph of a light distribution characteristic.

TABLE 3
Example 2

| Sn | R | D | Nd | νd |
|---|---|---|---|---|
| 1 | | 0.0803 | | |
| 2 | | 0.1497 | 1.4585 | 67.82 |
| 3 | ∞ | | | |

TABLE 4
Example 2

| | |
|---|---|
| A | 0.3662 |
| B | 0.600 |
| C | 0.0296 |
| G | 0.425 |
| K | 0.1497 |
| L | 0.230 |
| θ | 47 |
| ω | 82 |
| φ | 47 |
| α1 | 47 |
| β1 | 82 |
| Half-width at half-maximum | 42.5 |

### Example 3

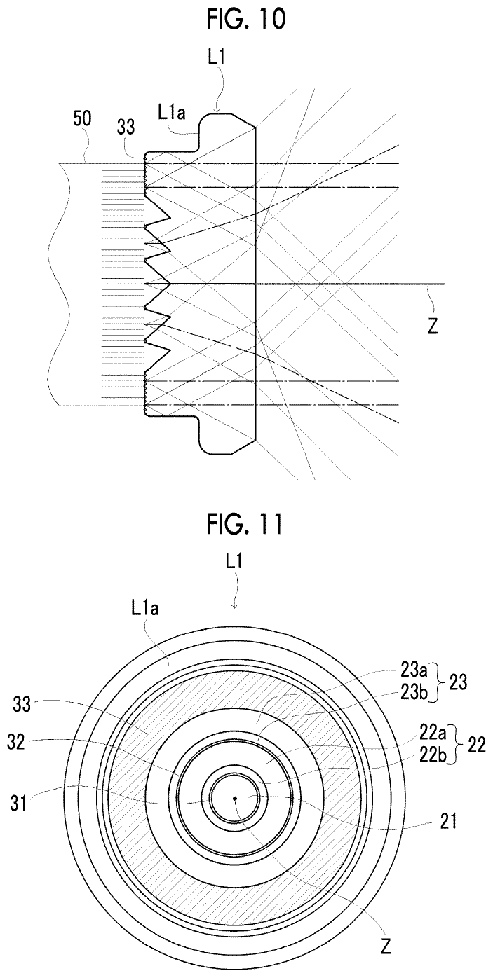

FIG. 10 is a cross-sectional view showing a configuration and an optical path of an illumination optical system of Example 3. FIG. 11 shows a configuration in a surface perpendicular to an optical axis Z of the illumination optical system of Example 3, viewed from the light source side. The illumination optical system of Example 3 has the same configuration as the outline of the illumination optical system of Example 1. In FIG. 10, some reference numerals are omitted in order to avoid complication of the drawing. The shape of the vicinity of the deepest portion of each groove portion of the illumination optical system of Example 3 is the same as that of the illumination optical system of Example 1. The illumination optical system of Example 3 is different from the illumination optical system of Example 1 in that the outer diameter of the light diffusion surface 33 is smaller than that of Example 1 and a step L1a is formed on the outer diameter side of the light diffusion surface 33. The surface perpendicular to the optical axis Z, which is included in the step L1a, is a surface that is not adjacent to the groove portion and is not a light diffusion surface.

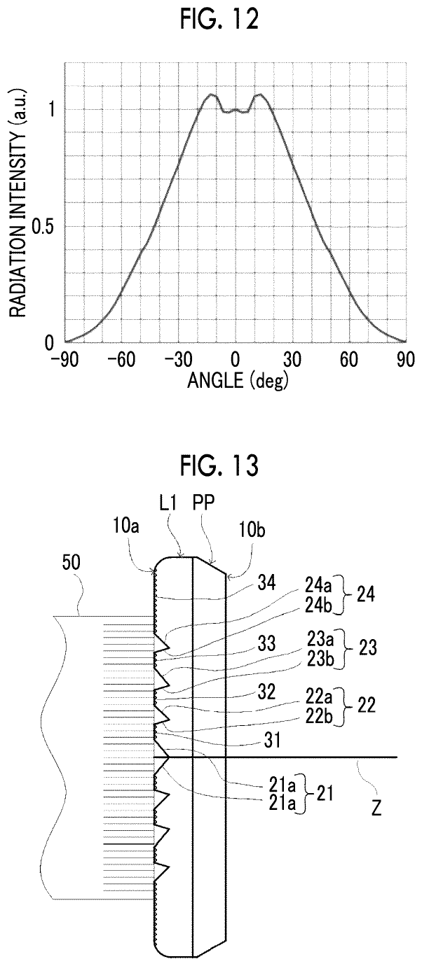

Regarding the illumination optical system of Example 3, Table 5 shows construction data, Table 6 shows specifications, and FIG. 12 shows a graph of a light distribution characteristic.

TABLE 5
Example 3

| Sn | R | D | Nd | νd |
|---|---|---|---|---|
| 1 | | 0.0900 | | |
| 2 | | 0.3000 | 1.4585 | 67.82 |
| 3 | ∞ | | | |

TABLE 6
Example 3

| | |
|---|---|
| A | 0.3139 |
| B | 0.600 |
| C | 0.0081 |
| G | 0.425 |
| K | 0.3000 |
| L | 0.390 |
| θ | 48 |
| ω | 73 |
| φ | 48 |
| α1 | 48 |
| β1 | 73 |
| Half-width at half-maximum | 42.8 |

### Example 4

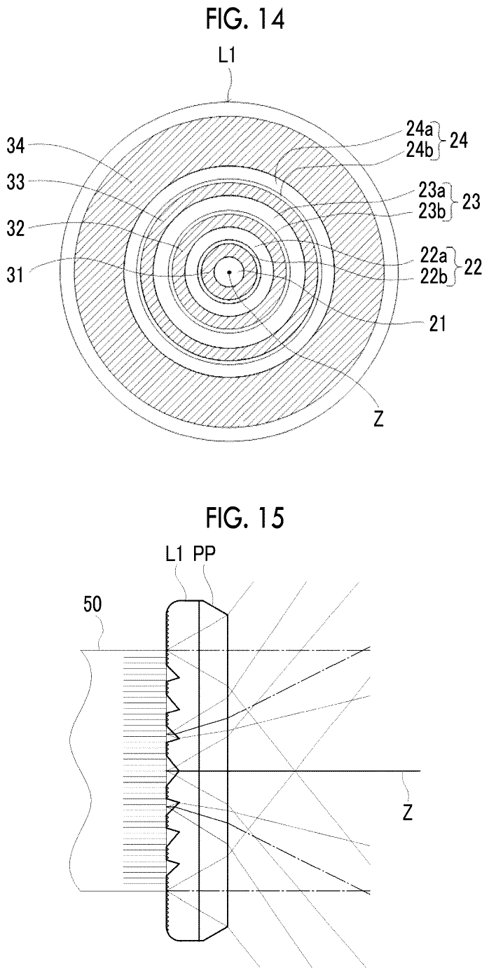

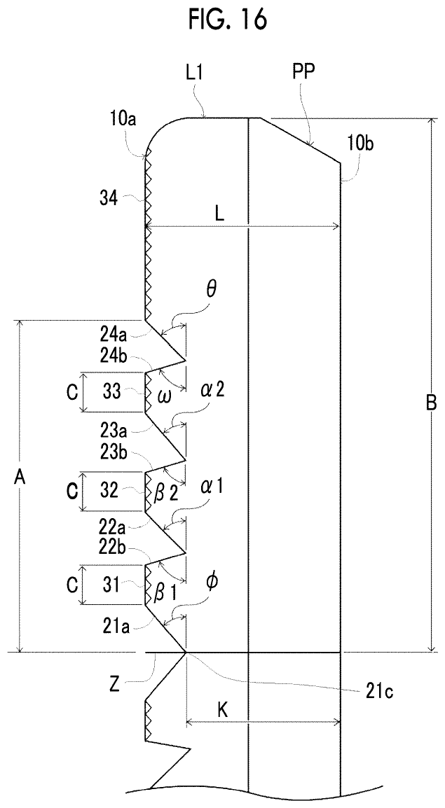

FIG. 13 shows a configuration of an illumination optical system of Example 4. FIG. 14 shows a configuration in a surface perpendicular to an optical axis Z of the illumination optical system of Example 4, viewed from the light source side. FIG. 15 is a cross-sectional view showing a configuration and an optical path of an illumination optical system of Example 4. FIG. 16 shows a partially enlarged view of an illumination optical system of Example 4.

The illumination optical system of Example 4 consists of one optical element L1 and one plane-parallel plate PP. The first surface 10a which is a surface closest to the light guide 50 side of the illumination optical system includes the groove portions 21, 22, 23, and 24 and the light diffusion surfaces 31, 32, 33, and 34. The groove portions 21, 22, 23, and 24 are ring-shaped grooves disposed in a concentric circular shape about the optical axis Z, and have a V-shape or a substantially V-shape in a cross section including the optical axis Z. The groove portion 21 has an inclined surface 21a. The groove portion 22 has an inclined surface 22a on the outer diameter side and an inclined surface 22b on the optical axis side. The groove portion 23 has an inclined surface 23a on the outer diameter side and an inclined surface 23b on the optical axis side. The groove portion 24 has an inclined surface 24a on the outer diameter side and an inclined surface 24b on the optical axis side. A light diffusion surface 31 is formed between the groove portion 21 and the groove portion 22 adjacent to the inclined surface 21a and the inclined surface 22b. A light diffusion surface 32 is formed between the groove portion 22 and the groove portion 23 adjacent to the inclined surface 22a and the inclined surface 23b. A light diffusion surface 33 is formed between the groove portion 23 and the groove portion 24 adjacent to the inclined surface 23a and the inclined surface 24b. A light diffusion surface 34 is formed on the outer diameter side adjacent to the inclined surface 24a of the groove portion 24 on the most outer diameter side. The light diffusion surfaces 31, 32, 33, and 34 are surfaces perpendicular to the optical axis Z, and the positions of the light diffusion surfaces 31, 32, 33, and 34 in the optical axis direction are the same. An outline of the illumination optical system of Example 4 is as described above. In Example 4, the light diffusion surfaces 31, 32, and 33 are the first light diffusion surfaces, and the light diffusion surface 34 is the second light diffusion surface. In the illumination optical system of Example 4, in a cross section including the optical axis Z, the vicinity of the deepest portion 21c (see FIG. 16) of the groove portion 21 has an apex angle, and the vicinity of the deepest portion of the groove portions 22, 23, and 24 has an acute angle shape.

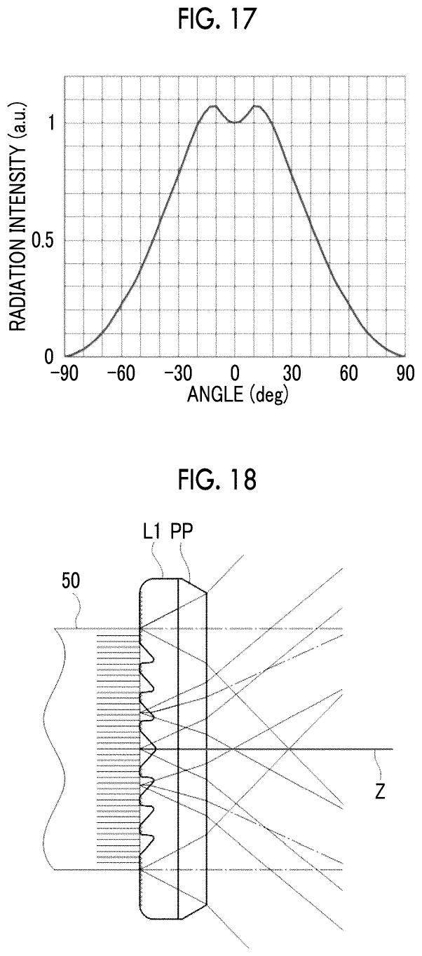

Regarding the illumination optical system of Example 4, Table 7 shows construction data, Table 8 shows specifications, and FIG. 17 shows a graph of a light distribution characteristic. In the illumination optical system consisting of one optical element L1 and one plane-parallel plate PP, a surface with surface number 4 corresponds to a surface 10b of the illumination optical system closest to the irradiation target side. Table 8 shows each value used in the above-described Conditional Expressions and the values of the angles α1, β1, α2, and β2 shown in FIG. 16. α1 is an angle formed between the inclined surface 22a of the groove portion 22 on the outer diameter side and the surface perpendicular to the optical axis Z. β1 is an angle formed between the inclined surface 22b of the groove portion 22 on the optical axis side and the surface perpendicular to the optical axis Z. α2 is an angle formed between the inclined surface 23a of the groove portion 23 on the outer diameter side and the surface perpendicular to the optical axis Z. β2 is an angle formed between the inclined surface 23b of the groove portion 23 on the optical axis side and the surface perpendicular to the optical axis Z.

TABLE 7
Example 4

| Sn | R | D | Nd | νd |
|---|---|---|---|---|
| 1 | | 0.0450 | | |
| 2 | | 0.0700 | 1.5188 | 54.00 |
| 3 | ∞ | 0.1000 | 1.5592 | 53.90 |
| 4 | ∞ | | | |

TABLE 8
Example 4

| | |
|---|---|
| A | 0.3734 |
| B | 0.600 |
| C | 0.0452 |
| G | 0.425 |
| K | 0.1700 |
| L | 0.215 |
| θ | 45 |
| ω | 73 |
| φ | 40 |
| α1 | 45 |
| β1 | 73 |
| α2 | 40 |
| β2 | 73 |
| Half-width at half-maximum | 43.5 |

### Example 5

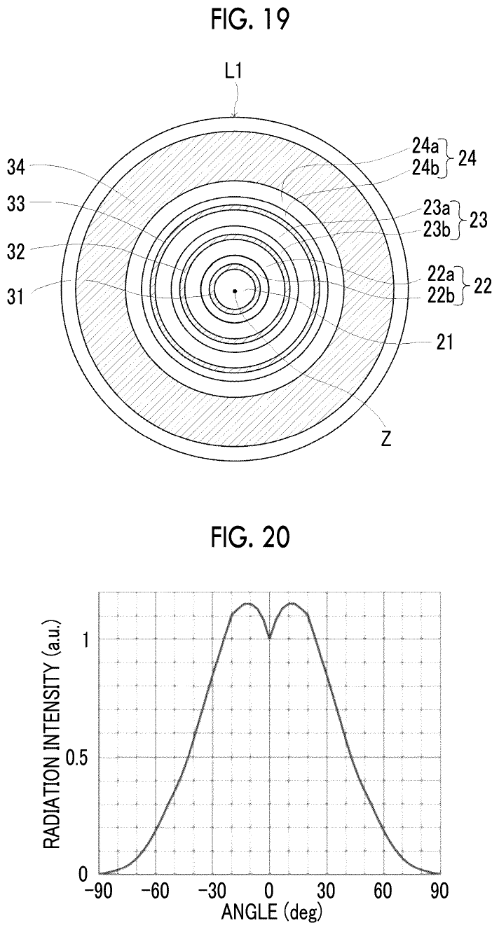

FIG. 18 is a cross-sectional view showing a configuration and an optical path of an illumination optical system of Example 5. FIG. 19 shows a configuration in a surface perpendicular to an optical axis Z of the illumination optical system of Example 5, viewed from the light source side. The illumination optical system of Example 5 has the same configuration as the outline of the illumination optical system of Example 4. In FIG. 18, some reference numerals are omitted in order to avoid complication of the drawing. In the illumination optical system of Example 5, the vicinity of the deepest portion of the groove portions 21, 22, 23, and 24 has a shape having a curvature.

Regarding the illumination optical system of Example 5, Table 9 shows construction data, Table 10 shows specifications, and FIG. 20 shows a graph of a light distribution characteristic.

TABLE 9
Example 5

| Sn | R | D | Nd | νd |
|---|---|---|---|---|
| 1 | | 0.0566 | | |
| 2 | | 0.0784 | 1.5188 | 54.00 |
| 3 | ∞ | 0.1000 | 1.5592 | 53.90 |
| 4 | ∞ | | | |

TABLE 10
Example 5

| | |
|---|---|
| A | 0.3777 |
| B | 0.600 |
| C | 0.0178 |
| G | 0.425 |
| K | 0.1784 |
| L | 0.235 |
| θ | 42 |
| ω | 85 |
| φ | 42 |
| α1 | 42 |
| β1 | 85 |
| α2 | 42 |
| β2 | 85 |
| Half-width at half-maximum | 43.0 |

### Example 6

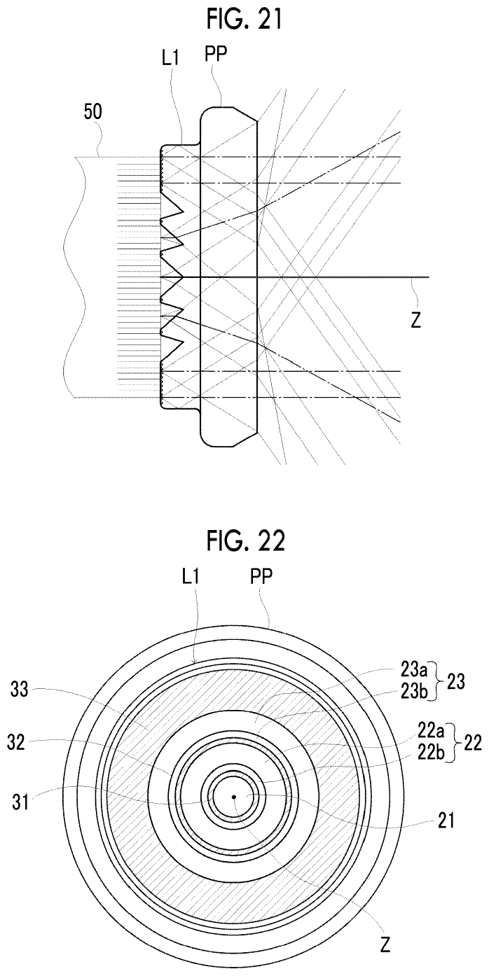

FIG. 21 is a cross-sectional view showing a configuration and an optical path of an illumination optical system of Example 6. FIG. 22 shows a configuration in a surface perpendicular to an optical axis Z of the illumination optical system of Example 6, viewed from the light source side. The illumination optical system of Example 6 consists of one optical element L1 and one plane-parallel plate PP. The outer diameter of the optical element L1 is smaller than the outer diameter of the plane-parallel plate PP. The outer shape of the entire illumination optical system of Example 6 has a step, similarly to the outer shape of the illumination optical system of Example 3. The surface of the optical element L1 on the light guide 50 side of Example 6 has the same configuration as the outline of the surface of the optical element L1 on the light guide 50 side of Example 3. In FIG. 21, some reference numerals are omitted in order to avoid complication of the drawing.

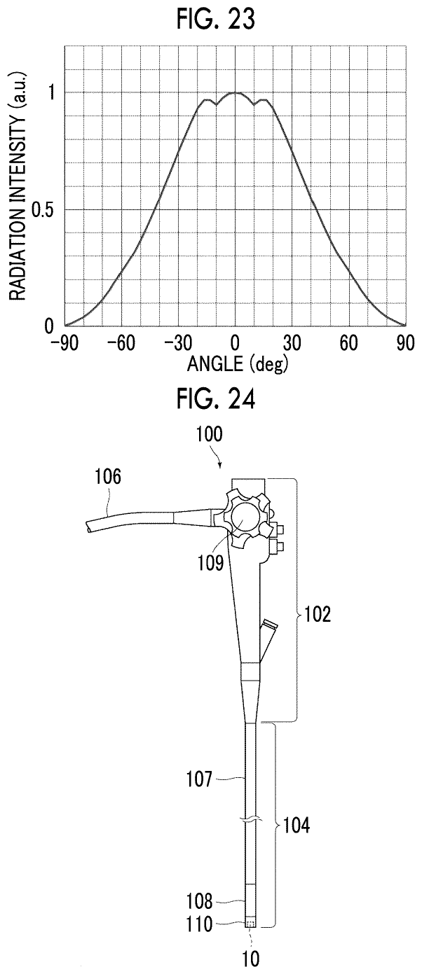

Regarding the illumination optical system of Example 6, Table 11 shows construction data, Table 12 shows specifications, and FIG. 23 shows a graph of a light distribution characteristic.

TABLE 11
Example 6

| Sn | R | D | Nd | νd |
|---|---|---|---|---|
| 1 | | 0.0800 | | |
| 2 | | 0.0600 | 1.5188 | 54.00 |
| 3 | ∞ | 0.2000 | 1.5592 | 53.90 |
| 4 | ∞ | | | |

TABLE 12
Example 6

| | |
|---|---|
| A | 0.3019 |
| B | 0.600 |
| C | 0.0186 |
| G | 0.425 |
| K | 0.2600 |
| L | 0.340 |
| θ | 48 |
| ω | 73 |
| φ | 48 |
| α1 | 48 |
| β1 | 73 |
| Half-width at half-maximum | 42.7 |

The corresponding values of Conditional Expressions (1) to (7) of the illumination optical systems of Examples 1 to 6 are shown in Table 13. Preferable ranges of the conditional expressions may be set using the corresponding values of the Examples shown in table 13 as upper limit values or lower limit values of the Conditional Expressions.

TABLE 13

| Expression | Conditional | Example | Example | Example | Example | Example | Example |
|---|---|---|---|---|---|---|---|
| number | Expression | 1 | 2 | 3 | 4 | 5 | 6 |
| (1) | C/A | 0.135 | 0.081 | 0.026 | 0.121 | 0.047 | 0.062 |
| (2) | (B − A)/ | 1.01 | 0.95 | 0.66 | 1.05 | 1.05 | 0.79 |
| | (L × tanθ) | | | | | | |
| (3) | K/L | 0.65 | 0.65 | 0.77 | 0.79 | 0.76 | 0.76 |
| (4) | (L − K)/ | 0.21 | 0.20 | 0.26 | 0.14 | 0.17 | 0.24 |
| | (A × tanφ) | | | | | | |
| (5) | G/A | 1.24 | 1.16 | 1.35 | 1.14 | 1.13 | 1.41 |
| (6) | \|ω − θ\| | 30 | 35 | 25 | 28 | 43 | 25 |
| (7) | B/A | 1.75 | 1.64 | 1.91 | 1.61 | 1.59 | 1.99 |

As described above, all of the illumination optical systems of Examples 1 to 6 are configured to be small, have a wide light distribution angle, and have a good transmission efficiency.

Next, an endoscope according to the embodiment of the present disclosure will be described. FIG. 24 is a schematic overall configuration view of the endoscope according to the embodiment of the present disclosure. An endoscope 100 shown in FIG. 24 mainly comprises an operating portion 102, an insertion portion 104, and a universal cord 106 that is to be connected to a connector portion (not shown). A large portion of the insertion portion 104 is a soft portion 107 that bends in any direction along an insertion path, a bendable portion 108 is connected to a distal end of the soft portion 107, and a distal end portion 110 is connected to a distal end of the bendable portion 108. The bendable portion 108 is provided to allow the distal end portion 110 to face a desired direction and can be operated to be bent by the rotational movement of a bending operation knob 109 provided at the operating portion 102.

The illumination optical system 10 according to the embodiment of the present disclosure is provided in a distal end of the distal end portion 110. The illumination optical system 10 is schematically shown in FIG. 24. Since the endoscope according to the embodiment of the present disclosure comprises the illumination optical system according to the embodiment of the present disclosure, it is possible to perform observation using good-quality illumination light with a wide angle while reducing the size of a distal end portion of the insertion portion 104.

The technique of the present disclosure has been hitherto described through embodiments and examples, but the technique of the present disclosure is not limited to the above-mentioned embodiments and examples, and may be modified into various forms. For example, values such as the thickness, the surface distance, the refractive index, and the Abbe number of each constituent are not limited to the values shown in the examples, and different values may be used therefor.

In the above description, an example in which the illumination optical system includes three or four groove portions has been described, but in the technology of the present disclosure, the number of groove portions included in the illumination optical system may be different from the number in the above example. Various modifications can be also made for the groove portions without departing from the scope of the present disclosed technology.

Regarding the above-described embodiments and examples, the following Supplementary Notes will be further disclosed.

### Supplementary Note 1

An illumination optical system that is disposed at a distal end of a light guide of an endoscope, the illumination optical system comprising:
a first surface that is a surface of the illumination optical system closest to a light guide side, the first surface including a plurality of ring-shaped groove portions that are arranged in a concentric circular shape about an optical axis of the illumination optical system,
wherein the groove portions have an inclined surface that narrows a groove width from the light guide side toward an irradiation target side,
in the first surface, all surfaces that are perpendicular to the optical axis and adjacent to the inclined surfaces of the groove portions are light diffusion surfaces,
the light diffusion surfaces include first light diffusion surfaces that are present between the groove portions adjacent to each other, and
in a case where a radial length of the first light diffusion surface is denoted by C and
a distance from the optical axis to a position of an outermost diameter of the groove portion on a most outer diameter side is denoted by A,

Conditional Expression (1) is satisfied, which is represented by

$$0.01 < C / A < 0.3 . \qquad ( 1 )$$

### Supplementary Note 2

The illumination optical system according to Supplementary Note 1,
wherein the light diffusion surfaces include a second light diffusion surface adjacent to the inclined surface on an outer diameter side of the groove portion on the most outer diameter side.

### Supplementary Note 3

The illumination optical system according to Supplementary Note 2,
wherein, in a case where a radius of the illumination optical system is denoted by B,
a distance in a direction of the optical axis from the second light diffusion surface to an intersection between the optical axis and a surface of the illumination optical system closest to the irradiation target side is denoted by L, and
an angle formed between the inclined surface on the outer diameter side of the groove portion on the most outer diameter side and the surface perpendicular to the optical axis is θ,

Conditional Expression (2) is satisfied, which is represented by

$$0.5 < ( B - A ) / ( L \times tan θ ) < 1.5 . \qquad ( 2 )$$

### Supplementary Note 4

The illumination optical system according to Supplementary Note 2 or 3,
wherein, in a case where a distance in a direction of the optical axis from the second light diffusion surface to an intersection between the optical axis and a surface of the illumination optical system closest to the irradiation target side is denoted by L, and
a distance in the direction of the optical axis from a deepest portion of the groove portion closest to the optical axis to the intersection is denoted by K,

Conditional Expression (3) is satisfied, which is represented by

$$0.5 < K / L < 0.88 . \qquad ( 3 )$$

### Supplementary Note 5

The illumination optical system according to any one of Supplementary Notes 2 to 4,
wherein, in a case where a distance in a direction of the optical axis from the second light diffusion surface to an intersection between the optical axis and a surface of the illumination optical system closest to the irradiation target side is denoted by L,
a distance in the direction of the optical axis from a deepest portion of the groove portion closest to the optical axis to the intersection is denoted by K, and
an angle formed between the inclined surface of the groove portion closest to the optical axis and the surface perpendicular to the optical axis is denoted by q,

Conditional Expression (4) is satisfied, which is represented by

$$0.104 < ( L - K ) / ( A \times tan φ ) < 0.4 . \qquad ( 4 )$$

### Supplementary Note 6

The illumination optical system according to any one of Supplementary Notes 1 to 5,
wherein, in a case where a radius of an emission end surface of the light guide is denoted by G,

Conditional Expression (5) is satisfied, which is represented by

$$1 < G / A < 1.8 . \qquad ( 5 )$$

### Supplementary Note 7

The illumination optical system according to any one of Supplementary Notes 1 to 6,
wherein, in a case where an angle formed between the inclined surface on an outer diameter side of the groove portion on the most outer diameter side and the surface perpendicular to the optical axis is denoted by θ,
an angle formed between the inclined surface on an optical axis side of the groove portion on the most outer diameter side and the surface perpendicular to the optical axis is denoted by ω, and
units of θ and ω are degrees,

Conditional Expression (6) is satisfied, which is represented by

$$20 < \vert ω - θ \vert < 50 . \qquad ( 6 )$$

### Supplementary Note 8

The illumination optical system according to any one of Supplementary Notes 1 to 7, wherein, in a case where a radius of the illumination optical system is denoted by B,

Conditional Expression (7) is satisfied, which is represented by

$$1.2 < B / A < 2.5 . \qquad ( 7 )$$

### Supplementary Note 9

An endoscope comprising: the illumination optical system according to any one of Supplementary Notes 1 to 8.

What is claimed is:

1. An illumination optical system that is disposed at a distal end of a light guide of an endoscope, the illumination optical system comprising:
a first surface that is a surface of the illumination optical system closest to a light guide side, the first surface including a plurality of ring-shaped groove portions that are arranged in a concentric circular shape about an optical axis of the illumination optical system,
wherein the groove portions have an inclined surface that narrows a groove width from the light guide side toward an irradiation target side,
in the first surface, all surfaces that are perpendicular to the optical axis and adjacent to the inclined surfaces of the groove portions are light diffusion surfaces,
the light diffusion surfaces include first light diffusion surfaces that are present between the groove portions adjacent to each other, and
in a case where a radial length of the first light diffusion surface is denoted by C and
a distance from the optical axis to a position of an outermost diameter of the groove portion on a most outer diameter side is denoted by A,
Conditional Expression (1) is satisfied, which is represented by
$$0.01 < C / A < 0.3 . \qquad ( 1 )$$

2. The illumination optical system according to claim 1,
wherein the light diffusion surfaces include a second light diffusion surface adjacent to the inclined surface on an outer diameter side of the groove portion on the most outer diameter side.

3. The illumination optical system according to claim 2,
wherein, in a case where a radius of the illumination optical system is denoted by B,
a distance in a direction of the optical axis from the second light diffusion surface to an intersection between the optical axis and a surface of the illumination optical system closest to the irradiation target side is denoted by L, and
an angle formed between the inclined surface on the outer diameter side of the groove portion on the most outer diameter side and the surface perpendicular to the optical axis is θ,
Conditional Expression (2) is satisfied, which is represented by
$$0.5 < ( B - A ) / ( L \times tan θ ) < 1.5 . \qquad ( 2 )$$

4. The illumination optical system according to claim 2,
wherein, in a case where a distance in a direction of the optical axis from the second light diffusion surface to an intersection between the optical axis and a surface of the illumination optical system closest to the irradiation target side is denoted by L, and
a distance in the direction of the optical axis from a deepest portion of the groove portion closest to the optical axis to the intersection is denoted by K,
Conditional Expression (3) is satisfied, which is represented by
$$0.5 < K / L < 0.88 . \qquad ( 3 )$$

5. The illumination optical system according to claim 2,
wherein, in a case where a distance in a direction of the optical axis from the second light diffusion surface to an intersection between the optical axis and a surface of the illumination optical system closest to the irradiation target side is denoted by L,
a distance in the direction of the optical axis from a deepest portion of the groove portion closest to the optical axis to the intersection is denoted by K, and
an angle formed between the inclined surface of the groove portion closest to the optical axis and the surface perpendicular to the optical axis is denoted by q,
Conditional Expression (4) is satisfied, which is represented by
$$0.104 < ( L - K ) / ( A \times tan φ ) < 0.4 . \qquad ( 4 )$$

6. The illumination optical system according to claim 1,
wherein, in a case where a radius of an emission end surface of the light guide is denoted by G,
Conditional Expression (5) is satisfied, which is represented by
$$1 < G / A < 1.8 . \qquad ( 5 )$$

7. The illumination optical system according to claim 1,
wherein, in a case where an angle formed between the inclined surface on an outer diameter side of the groove portion on the most outer diameter side and the surface perpendicular to the optical axis is denoted by θ,
an angle formed between the inclined surface on an optical axis side of the groove portion on the most outer diameter side and the surface perpendicular to the optical axis is denoted by ω, and
units of θ and ω are degrees,
Conditional Expression (6) is satisfied, which is represented by
$$20 < \vert ω - θ \vert < 50 . \qquad ( 6 )$$

8. The illumination optical system according to claim 1,
wherein, in a case where a radius of the illumination optical system is denoted by B,
Conditional Expression (7) is satisfied, which is represented by
$$1.2 < B / A < 2.5 . \qquad ( 7 )$$

9. An endoscope comprising: the illumination optical system according to claim 1.
